(19) United States
(12) Patent Application Publication
(10) Pub. No.: US 2022/0181010 A1
(43) Pub. Date: Jun. 9, 2022

(54) MEDICAL CARE SUPPORT DEVICE

(71) Applicant: FUJIFILM Corporation, Tokyo (JP)

(72) Inventors: Tsuyoshi HIRAKAWA, Tokyo (JP); Hiroshi HIRAMATSU, Tokyo (JP); Keiji TSUBOTA, Tokyo (JP); Misaki KAWAHARA, Tokyo (JP)

(73) Assignee: FUJIFILM Corporation, Tokyo (JP)

(21) Appl. No.: 17/677,918

(22) Filed: Feb. 22, 2022

Related U.S. Application Data
(63) Continuation of application No. PCT/JP2020/021865, filed on Jun. 3, 2020.

(30) Foreign Application Priority Data
Sep. 27, 2019 (JP) 2019-177804

(51) Int. Cl. G16H 40/20; H04L 65/4038; G10L 15/22; G10L 15/26

(52) U.S. Cl. CPC G16H 40/20; H04L 65/4038; G16H 10/60; G10L 15/26; G10L 15/22

(57) ABSTRACT

A conference information management unit generates, for each of conferences scheduled to be executed, conference information including information on the type, target patients, participants, and execution date and time. A participant job type determination unit compares a job type definition table with the conference information to determine whether or not the participants of the job type defined in the job type definition table are included in the conference scheduled to be executed. The determination result is notified by the conference information management unit.

### CROSS-REFERENCE TO RELATED APPLICATIONS

This application is a Continuation of PCT International Application No. PCT/JP2020/021865 filed on Jun. 3, 2020, which claims priority under 35 U.S.C § 119(a) to Japanese Patent Application No. 2019-177804 filed on Sep. 27, 2019. Each of the above application(s) is hereby expressly incorporated by reference, in its entirety, into the present application.

### BACKGROUND OF THE INVENTION

### 1. Field of the Invention

The present invention relates to a medical care support device.

### 2. Description of the Related Art

The medical staff has a wide range of duties, and conferences are one of the duties. In order to reduce the burden on busy medical staff, a technique for efficiently searching and displaying necessary examination information or the like during a conference is disclosed. For example, JP2016-018491A specifies a target patient for each type of the conferences and generates conference materials in which medical information regarding the target patient is aggregated. Further, JP2013-186651A generates a conference list in which the name of the conference is associated with a target patient and participants.

### SUMMARY OF THE INVENTION

In recent years, team medical care has been promoted in which many job types such as doctors, nurses, various technicians (engineers), rehabilitation staff, pharmacists, registered dietitians, and the like cooperate. Conferences in team medical care are diverse with agenda items such as nutritional management, pressure ulcer control, swallowing, and rehabilitation. In such a multidisciplinary conference, it is necessary to set the conference without omission and efficiently manage the conference such as notifying the participants of the conference of participation or holding or registering as a participant in the set conference.

An object of the present disclosure is to provide a medical care support device capable of efficiently extracting participant candidates of conferences scheduled to be executed and managing conference information regarding the conferences scheduled to be executed in a medical conference.

In order to solve the above problems, according to an aspect of the present disclosure, there is provided a medical care support device comprising a conference information management unit that generates, for each of conferences scheduled to be executed, conference information including information on a type of the conference, target patients for the conference, participants of the conference, and an execution date and time of the conference, and a participant job type determination unit that compares a job type definition table in which participant job types for each type of the conferences are defined with the conference information to determine whether or not the participants of the job type defined in the job type definition table are included in the conference scheduled to be executed, in which the conference information management unit gives a notification of a determination result of the participant job type determination unit.

It is preferable that the medical care support device further comprises a display screen generation unit that generates a conference list display screen in which the conferences scheduled to be executed are listed.

It is preferable that the display screen generation unit generates a conference information display screen that displays the conference information of the conference selected from the conference list display screen.

It is preferable that the conference information management unit receives a change operation for changing the conference information displayed on the conference information display screen, and changes the conference information to the received contents.

It is preferable that the job type definition table is associated with participant job type importance, which is importance of the participant job type for each type of the conferences.

It is preferable that the conference information management unit extracts the target patient for each type of the conferences by using medical care information related to medical care of patients, and generates the conference information of the conference for the extracted target patient.

It is preferable that the conference information management unit extracts candidates of the participants for each type of the conferences by using the job type definition table, and transmits a participation registration notification to the candidates of the participants.

It is preferable that the conferences are categorized by medical care processes, and the participant job type includes a doctor and a nurse.

It is preferable that the medical care support device further comprises a voice recognition unit that recognizes a voice input from a voice input unit, a medical care data acquisition unit that, in a case where the voice recognized by the voice recognition unit during the execution of the conference includes an instruction to transmit medical care data acquired in medical care of a target patient of the conference being executed, accesses a medical care data storage unit in which the medical care data is stored and acquires the medical care data, and a medical care data display screen generation unit that generates a medical care data display screen including the medical care data acquired by the medical care data acquisition unit.

It is preferable that the medical care support device further comprises a voice recognition unit that recognizes a voice input from a voice input unit, a voice conversion unit that converts the voice recognized by the voice recognition unit during the execution of the conference into text information, and a minutes creation unit that creates minutes of the conference being executed by using the text information converted by the voice conversion unit.

It is preferable that the medical care support device further comprises a voice recognition unit that recognizes a voice input from a voice input unit, a voice conversion unit that converts the voice recognized by the voice recognition unit during the execution of the conference into text information, and a minutes creation unit that creates minutes of the conference being executed by using the text information converted by the voice conversion unit.

It is preferable that the medical care support device further comprises a registration unit that registers the minutes of the conference in association with an electronic medical record of the target patient.

According to the aspect of the present disclosure, by comparing the participant candidates of the conference scheduled to be executed with the job types defined in a job type definition table, and determining whether or not the participant job types recommended to participate in the conference scheduled to be executed are included, it is possible to efficiently manage the participants of the conference scheduled to be executed and the conference information regarding the conference.

### BRIEF DESCRIPTION OF THE DRAWINGS

FIG. 1 is a diagram showing a medical care support system.

FIG. 2 is a diagram showing the contents of an electronic medical record stored in a medical record database.

FIG. 3 is a diagram showing the contents of examination images stored in an image database.

FIG. 4 is a diagram showing the contents of a medical report stored in a report database.

FIG. 5 is a diagram showing a computer constituting a medical care support device or a client terminal.

FIG. 6 is a diagram showing a function of a CPU of a client terminal.

FIG. 7 is a diagram showing a conference-related information database and a medical care support device.

FIG. 8 is a diagram showing a conference execution schedule.

FIG. 9 is a diagram showing medical staff information.

FIG. 10 is a diagram showing a job type definition table.

FIG. 11 is a diagram showing a display screen displayed on a client terminal.

FIG. 12 is a diagram showing a flow of participation registration of conference participants.

FIG. 13 is a diagram showing extraction of target patients for a conference.

FIG. 14 is a diagram showing a clinical flow.

FIG. 15 is a diagram showing extraction of participant candidates for a conference.

FIG. 16 is a diagram showing a participation registration screen transmitted to a terminal of a participant candidate.

FIG. 17 is a diagram showing a change button in conference information.

FIG. 18 is a diagram showing participants of a conference.

FIG. 19 is a diagram showing a comparison between the job types of participants of the conference and the job type definition table.

FIG. 20 is a diagram showing re-notification to participant candidates of the conference.

FIG. 21 is a diagram showing the addition of medical staff other than participant candidates.

FIG. 22 is a diagram showing a medical care support system according to a second embodiment.

FIG. 23 is a diagram showing a medical care support system according to a third embodiment.

### DESCRIPTION OF THE PREFERRED EMBODIMENTS

### First Embodiment

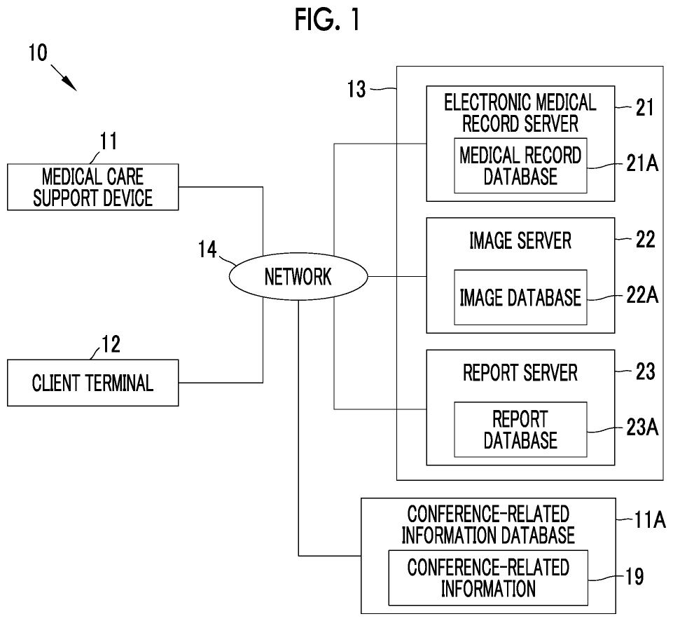

As shown in FIG. 1, a medical care support system 10 is a computer system for providing medical care support in a medical facility such as a hospital. The medical care support system 10 comprises a medical care support device 11, a client terminal 12, a server group 13, and a conference-related information database 11A. These are connected to each other so as to be able to communicate with each other by a network 14 such as a local area network (LAN) laid in the medical facility.

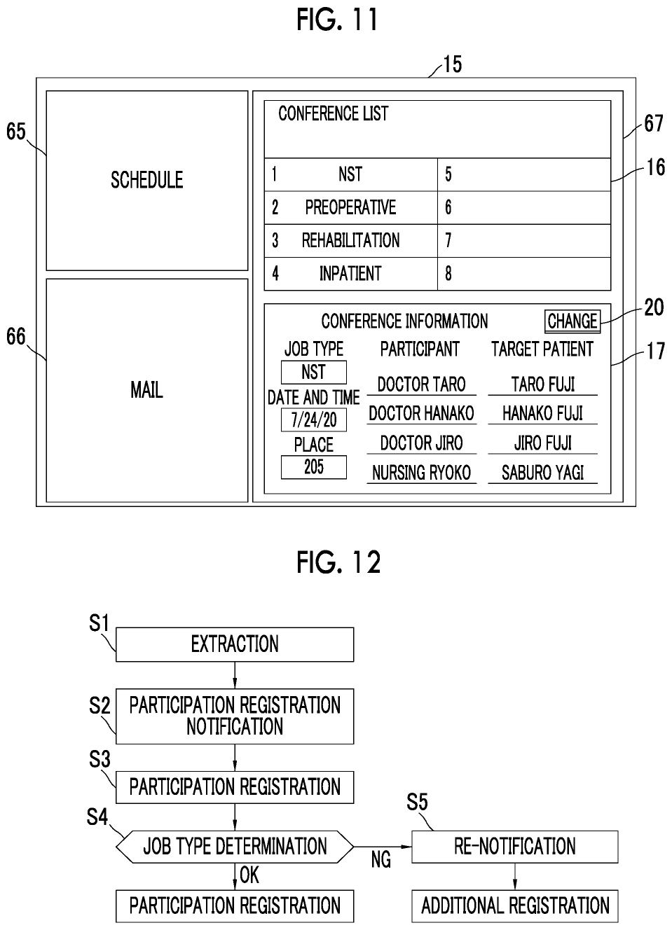

The medical care support device 11 extracts a target patient based on medical care data acquired in the medical care process of the patient, and generates conference information in which the target patient to be the agenda of the conference and the participants of the conference are associated with each other for each type of the conferences scheduled to be executed. The medical care support device 11 further generates a conference list display screen 16 and a conference information display screen 17 (see FIG. 11), and distributes a display screen 15 including the conference list display screen 16 (see FIG. 11) to the client terminal 12. The types of conferences are categorized by medical care processes, such as medical examinations and medical care tests performed by medical staff on patients.

The client terminal 12 is installed in each medical department in a medical facility such as internal medicine, surgery, otolaryngology, and ophthalmology, and in each examination department such as a radiological examination department or a clinical examination department and is operated by a doctor in each medical department, a laboratory technician in each examination department, or a medical staff such as a nurse or a registered dietitian. The client terminal 12 displays the display screen 15 transmitted from the medical care support device 11 and functions as a viewer terminal for medical staff to view.

The medical care support device 11 distributes the display screen 15 to the client terminal 12 in the format of extensible markup language (XML) data for web distribution created by a markup language such as XML. The client terminal 12 reproduces and displays the display screen 15 on the web browser based on the XML data. In addition, instead of XML, other data description languages such as JavaScript (registered trademark) Object Notation (JSON) may be used.

The server group 13 searches for medical care data corresponding to an acquisition request from the medical care support device 11, and transmits the searched medical care data to the medical care support device 11. The server group 13 comprises an electronic medical record server 21, an image server 22, and a report server 23.

The electronic medical record server 21 has a medical record database 21A in which an electronic medical record 24 is stored. In the electronic medical record 24, as medical care data, medical examination record data in which interview contents, diagnosis contents, disease names, etc. are recorded, examination data such as specimen tests such as blood tests and biochemical tests, and test values of medical care tests including physiological tests such as electrocardiogram tests and brain wave tests, measurement data such as measured values of vital signs such as a patient's heartbeat, pulse, blood pressure, and body temperature, order data in which various orders such as medical care test, creation, treatment, surgery, and medication are recorded, treatment record data in which events occurring in the patient's medical care process such as initial medical examination, hospitalization, discharge, rehospitalization, treatment, surgery, medication, and complete cure are recorded, accounting data in which consultation fees, drug fees, hospitalization fees, etc. are recorded are input. These various medical care data of the electronic medical record 24 can be input on the client terminal 12 and can be viewed on the client terminal 12.

The image server 22 is a so-called PACS (picture archiving and communication system) server, and has an image database 22A in which examination image 25 is stored. The examination image 25 is an image obtained by various image examinations such as computed tomography (CT) examination, magnetic resonance imaging (MRI) examination, simple X-ray examination, ultrasonography, and endoscopy, and is created, for example, in a digital imaging and communications in medicine (DICOM) standard data file format. The examination image 25 can be viewed on the client terminal 12.

The report server 23 has a report database 23A in which a medical report 26 is stored. The medical report 26 summarizes the results of interpretation of the examination image 25 obtained by the image examination by the radiologist. The medical report 26 can be created and viewed on the client terminal 12.

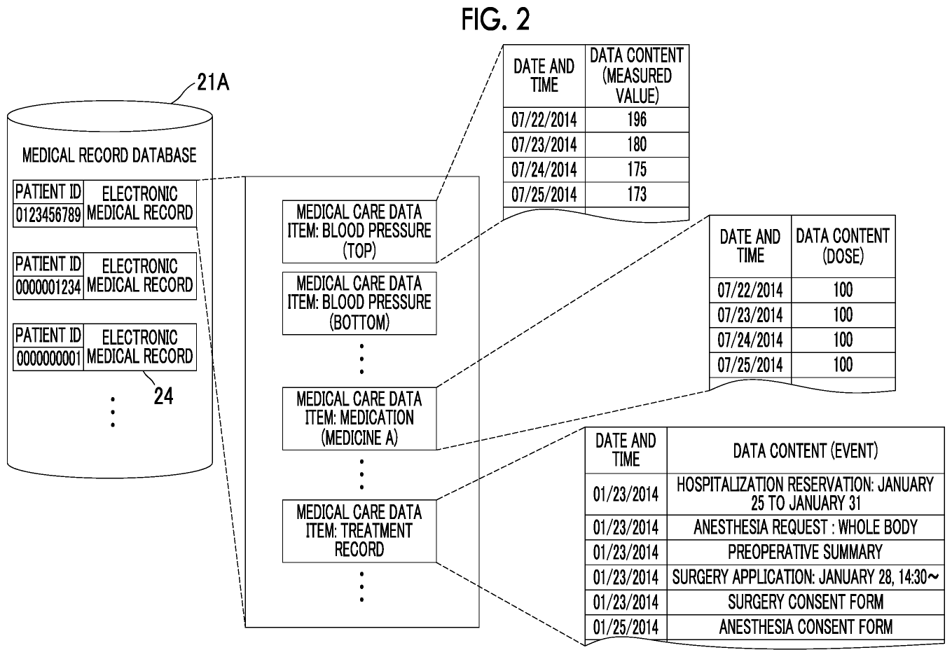

The electronic medical record 24, the examination image 25, and the medical report 26 store patient IDs (identification data) and the like, which are numbers and symbols for identifying individual patients, in association with each other as accessory information (FIGS. 2 to 4).

As shown in FIG. 2, the electronic medical record 24 stored in the medical record database 21A is managed on a patient-by-patient basis in association with a patient ID represented by a 10-digit number such as “0123456789”. In the electronic medical record 24, in addition to the patient ID, patient information such as the patient's name, gender, date of birth, or age, and medical care data of a plurality of items are recorded. The medical care data is organized by items such as “blood pressure (top)”, “blood pressure (bottom)”, “biochemical test A”, and “treatment record” and recorded in time series.

One record for each item of medical care data includes information on the date and time such as the medical care date and time, the examination date and time, the measurement date and time, the medication date and time (date and time of administration or prescription), and the date and time of various events occurring in the patient's medical care process, and data contents such as interview contents, diagnosis contents, test values, measured values, doses, medical fee amounts, and events.

The events recorded in the treatment record data include hospitalization, surgery, transfer, discharge, rehospitalization, etc., and the contents of the hospitalization reservation accompanying the hospitalization event, the contents of the anesthesia request accompanying the surgery event, the preoperative summary, the contents of the surgery application, the anesthesia consent form, the surgery consent form, etc. are also recorded. The preoperative summary is a summary of the results of medical care tests performed before surgery. Anesthesia consent form and surgery consent form are documents that explain the necessity and risk of anesthesia and surgery to patients and their families, and request the patients and their families to sign and seal.

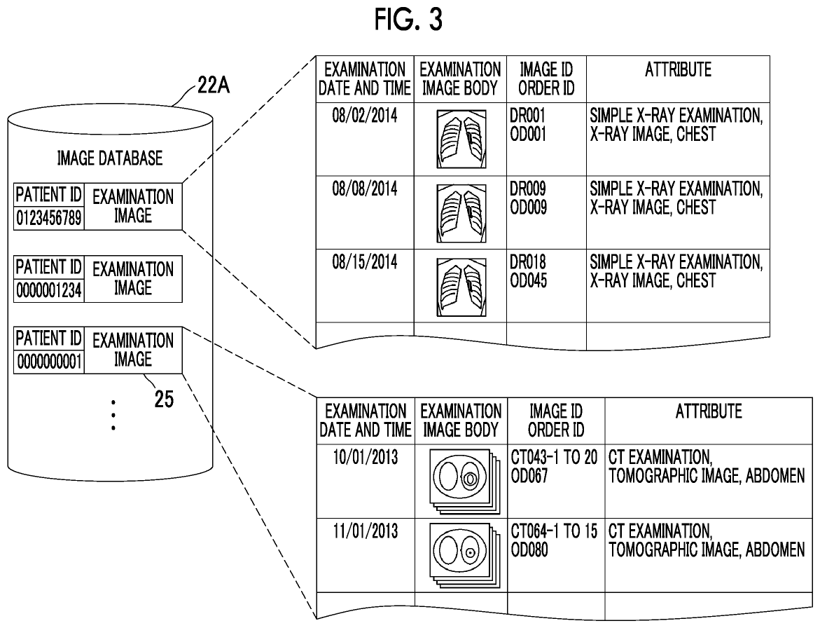

As shown in FIG. 3, the examination image 25 stored in the image database 22A is managed on a patient-by-patient basis in association with the patient ID as in the electronic medical record 24. In addition to the patient ID, the examination image 25 is associated with an examination date and time when the image examination was performed (a date and time when the examination image 25 was uploaded to the image database 22A), an image ID and an order ID, and attributes of the examination image 25 such as types of image examinations such as “simple X-ray examination” and “CT examination”, types of examination images such as “X-ray image” and “tomographic image”, and imaging parts such as “chest” and “abdomen” as accessory information. The image server 22 transmits the examination image 25 as medical care data to the medical care support device 11 together with accessory information such as the examination date and time and attributes.

The image ID is a number or symbol for identifying each examination image 25, and the order ID is a number or symbol for identifying each order. In a simple X-ray examination, one X-ray image is often taken in one image examination. On the other hand, a plurality of examination images 25 may be taken in one image examination, such as a tomographic image acquired by a CT examination. In a case where a plurality of examination images 25 are taken in one image examination in this way, a common order ID is assigned to each examination image 25 to show that a plurality of examination images 25 have been obtained in one image examination, and the examination images 25 are managed as a single examination image 25. The same applies to a case where a plurality of images are taken by a simple X-ray examination.

The accessory information of the examination image 25 may include information on the position and size of the lesion in the examination image 25, the type of the lesion, the feature amount of the lesion, the healing level of the lesion, and the like. In a case where the image examination is an ultrasonography, a blood flow measurement value obtained by analyzing an ultrasound image may be included as accessory information.

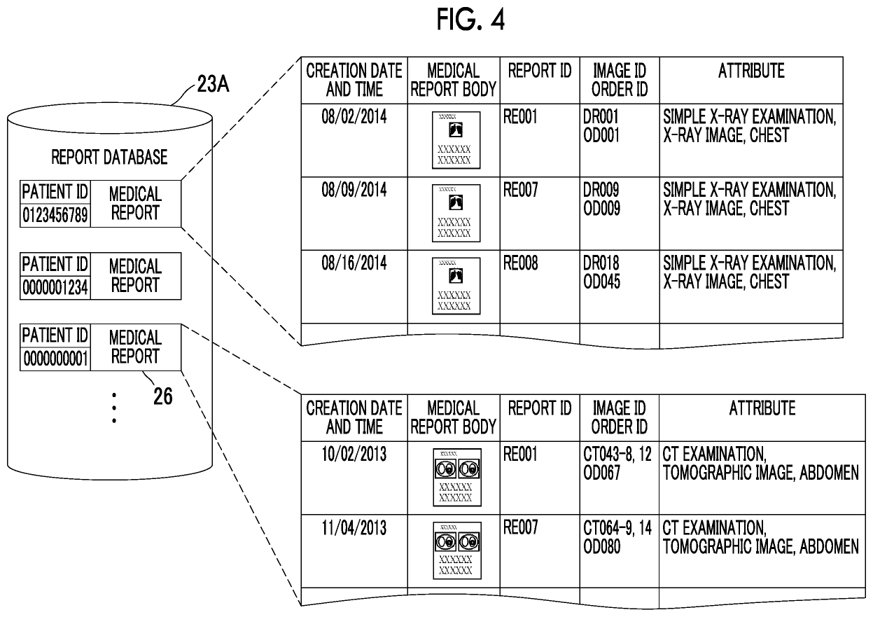

As shown in FIG. 4, the medical report 26 stored in the report database 23A is managed on a patient-by-patient basis in association with the patient ID as in the electronic medical record 24 and the examination image 25. In addition to the patient ID, the medical report 26 is associated with a creation date and time of the medical report 26 (a date and time when the medical report 26 was uploaded to the report database 23A), a report ID of the medical report 26, an image ID and an order ID of the examination image 25 attached to the medical report 26, and attributes similar to those of the examination image 25 as accessory information. The report server 23 transmits the medical report 26 as medical care data to the medical care support device 11 together with such accessory information. Note that FIGS. 2 to 4 show only dates among various dates and times.

The medical care support device 11, the client terminal 12, and the servers 21 to 23 of the server group 13 are configured by installing a control program such as an operating system or an application program such as a server program or a client program based on a computer such as a server computer, a personal computer, or a workstation.

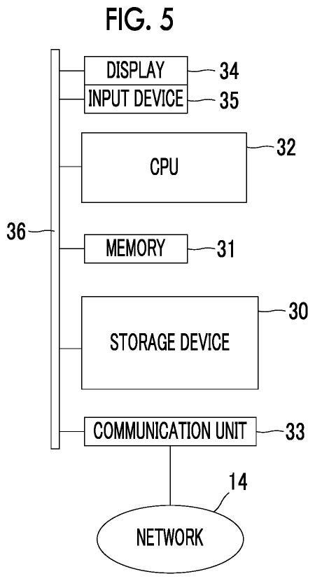

As shown in FIG. 5, the computers constituting the medical care support device 11, the client terminal 12, and the like have the same basic configuration, and comprise a storage device 30, a memory 31, a central processing unit (CPU) 32, a communication unit 33, a display 34, and an input device 35, respectively. These are interconnected via a data bus 36.

The storage device 30 is a hard disk drive built in the computer constituting the medical care support device 11, the client terminal 12, or the like or connected through a cable or a network, or a disk array in which a plurality of hard disk drives are connected. The storage device 30 stores control programs such as an operating system, various application programs, and display data of various operation screens associated with these programs.

The memory 31 is a work memory for the CPU 32 to execute processing. The CPU 32 generally controls each unit of the computer by loading the program stored in the storage device 30 into the memory 31 and executing the processing according to the program.

The communication unit 33 is a network interface for controlling transmission of various information via the network 14. The display 34 displays various operation screens according to the operation of the input device 35 such as a mouse and a keyboard. The operation screen is equipped with an operation function by a graphical user interface (GUI). The computer constituting the medical care support device 11, the client terminal 12, or the like receives the input of the operation instruction from the input device 35 through the operation screen. In the following description, the subscript “A” is attached to each unit of the computer constituting the medical care support device 11 and the subscript “B” is attached to each unit of the computer constituting the client terminal 12 to distinguish them.

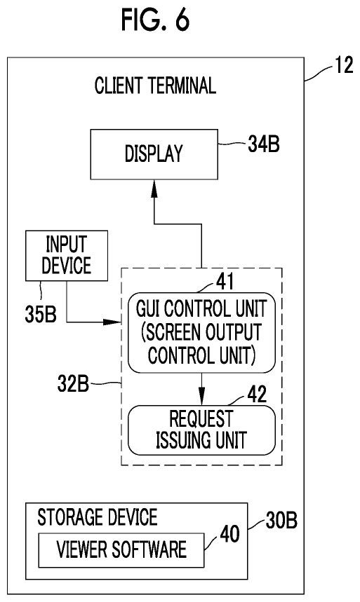

As shown in FIG. 6, a storage device 30B of the client terminal 12 stores viewer software 40 for viewing the display screen 15 as an application program. The viewer software 40 includes a plurality of gadget engines (hereinafter simply referred to as gadgets) that control the display of a plurality of display units constituting the display screen 15. The gadget is a subprogram that exhibits various functions by operating alongside a main application program such as the viewer software 40.

In a case where the viewer software 40 is activated, a CPU 32B of the client terminal 12 cooperates with a memory 31B (not shown) to function as a GUI control unit 41 and a request issuing unit 42.

The GUI control unit 41 displays the display screen 15 distributed from the medical care support device 11 on a web browser of a display 34B. The GUI control unit 41 controls the screen output of the display screen 15 in response to an operation instruction input from an input device 35B through the display screen 15, such as a button click operation with a mouse cursor (not shown).

The request issuing unit 42 issues various processing requests to the medical care support device 11 in response to an operation instruction of the input device 35B via the GUI control unit 41. The various processing requests include a distribution request and an edit request for the display screen 15. Various processing requests issued by the request issuing unit 42 are transmitted to the medical care support device 11 through the network 14.

The distribution request includes a medical staff ID. The medical staff ID is input through, for example, a login screen (not shown) for activating the display screen 15 together with an authentication key.

The edit request is for requesting the medical care support device 11 to edit the display contents of the display screen 15 in response to various operation instructions by the input device 35B.

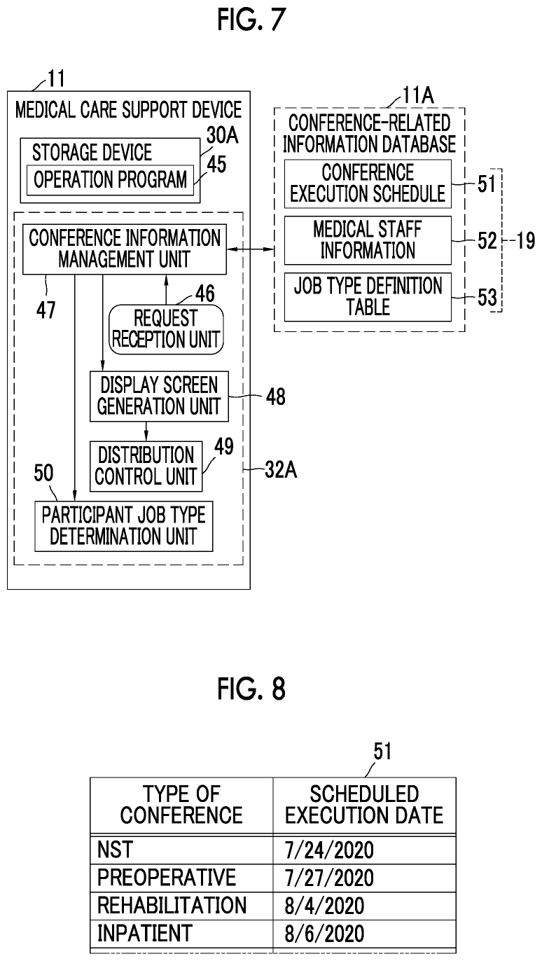

As shown in FIG. 7, an operation program 45 is stored as an application program in a storage device 30A of the medical care support device 11. The operation program 45 is a program for causing the computer constituting the medical care support device 11 to function as the medical care support device. In a case where the operation program 45 is activated, a CPU 32A of the medical care support device 11 cooperates with a memory 31A (not shown) to function as a request reception unit 46, a conference information management unit 47, a display screen generation unit 48, a distribution control unit 49, and a participant job type determination unit 50.

The request reception unit 46 receives a distribution request and an edit request from the client terminal 12. The request reception unit 46 outputs the received distribution request and edit request to the conference information management unit 47.

The conference information management unit 47 manages conference-related information 19 of the conference-related information database 11A. The conference-related information 19 includes a conference execution schedule 51 in which the scheduled execution date of the conference is recorded, medical staff information 52 in which the medical staff who participate in the conference are stored, and a job type definition table 53 in which the job types of the participants of the conference for each type of the conferences are defined.

As shown in FIG. 8, the conference execution schedule 51 is for determining the timing at which the conference information management unit 47 generates the conference information. As will be described later, the conference information is generated based on the scheduled execution date of the conference stored in the conference execution schedule 51. For example, in a case where the conference type is “NST”, the scheduled execution date is “2020/7/24”.

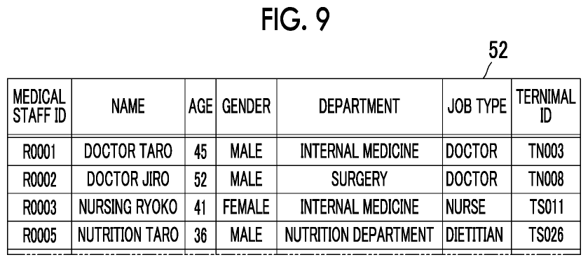

As shown in FIG. 9, the medical staff information 52 is managed on a medical staff-by-medical staff basis in association with the medical staff ID. The medical staff information 52 records basic information of the medical staff such as the name, age, gender, department, job type, and terminal ID of the medical staff. For example, as information associated with a medical staff ID “R0001”, the name is “Doctor Taro”, the age is “45 years old”, the gender is “male”, the department is “internal medicine”, the job type is “doctor”, and the terminal ID is “TN003”.

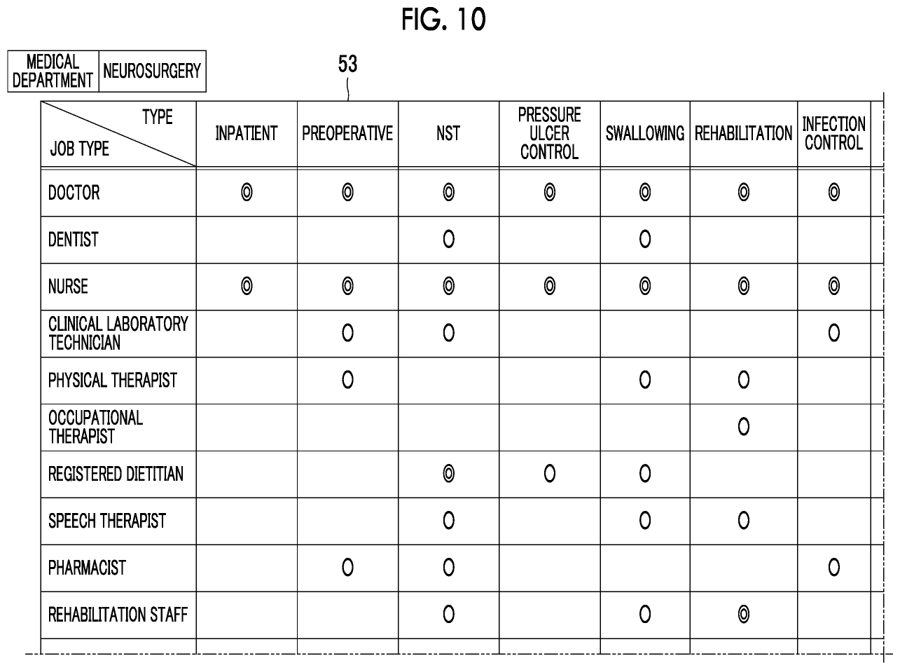

As shown in FIG. 10, the job type definition table 53 defines the participant job types for each type of the conferences. Types of conferences include inpatient conferences, preoperative conferences, nutrition support team (NST) conferences, pressure ulcer control conferences, swallowing conferences, rehabilitation conferences, and infection control conferences. Participants' job types include doctors, dentists, nurses, clinical laboratory technicians, physical therapists, occupational therapists, registered dietitians, speech therapists, pharmacists, and rehabilitation staff. NST is a type of team medical care that manages the nutrition of patients.

As for the job types of participants of the conference, participant job type importance, which is importance of the job types recommended to participate in the conference, is set in two stages. Participant job type importance is set as a job type that mainly engages in work corresponding to the type of conference, a job type that mainly engages in discussions at the conference, and the like, as a job type that is recommended to participate in the conference. As shown in FIG. 10, double circles (“⊚”) are job types that are required to participate, and circles (“◯”) are job types that are recommended to participate. For example, in a case where the type of conference is “NST”, participation is indispensable for the job types of doctors, nurses, and registered dietitians because the participant job type importance is “502 ”. For the job types of dentists, clinical laboratory technicians, speech therapists, pharmacists, and rehabilitation staff, participation is recommended because the participant job type importance is “◯”.

As shown in FIG. 7, the conference information management unit 47 accesses the server group 13 at a specific timing before the scheduled execution date of the conference recorded in the conference execution schedule 51, extracts target patients according to the type of conference, and further extracts participant candidates according to the type of conference from the medical staff information 52. The conference information management unit 47 generates conference information and a conference list by associating the extracted target patients and participant candidates with the types of conferences. Further, the conference information management unit 47 updates the medical staff information 52 in response to an edit request from the request reception unit 46.

As shown in FIG. 11, the display screen generation unit 48 generates the conference list display screen 16 for displaying the conference list and the conference information display screen 17 for displaying the conference information. Further, the display screen generation unit 48 generates and edits the display screen 15 including the conference list display screen 16 based on the edit request received by the request reception unit 46.

The distribution control unit 49 controls the display screen 15 to be distributed to the client terminal 12 that has transmitted the processing request via the communication unit 33A (not shown).

As shown in FIG. 11, the display screen 15 has three display units 65 to 67: a schedule display unit 65 in the upper left, a mail display unit 66 in the lower left, and a conference information display unit 67 on the right side. The display positions, display areas, and display contents of these three display units 65 to 67 can be freely customized by medical staff.

The schedule display unit 65 is generated by the schedule management gadget. Further, the mail display unit 66 is generated by the mail management gadget. The schedule management gadget and the mail management gadget are a kind of gadgets included in the viewer software 40. The schedule displayed on the schedule display unit 65 and the content of the e-mail displayed on the mail display unit 66 are switched according to the medical staff ID inputted in the login screen.

The conference list display screen 16 and the conference information display screen 17 are displayed on the conference information display unit 67. As shown in the conference list display screen 16, the conference information display screen 17 displays the conference information associated with the conference selected from the conference list. The conference information includes a list of target patients extracted by the conference information management unit 47 and a list of participants who have registered for participation. For example, in the case of selecting NST conference of 1 from the conference lists 1 to 8 displayed on the conference list display screen 16, “1 NST” is clicked. Thereby, on the conference information display screen 17, the date and time, the place, the participant, and the target patient are displayed as the conference information of the selected NST conference of 1.

Here, the generation of the conference list and the conference information will be described using the flowchart of FIG. 12 by taking the NST conference as an example.

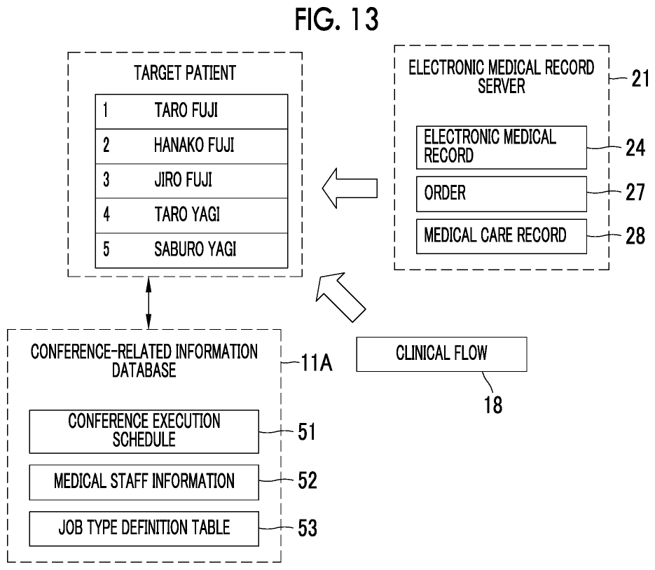

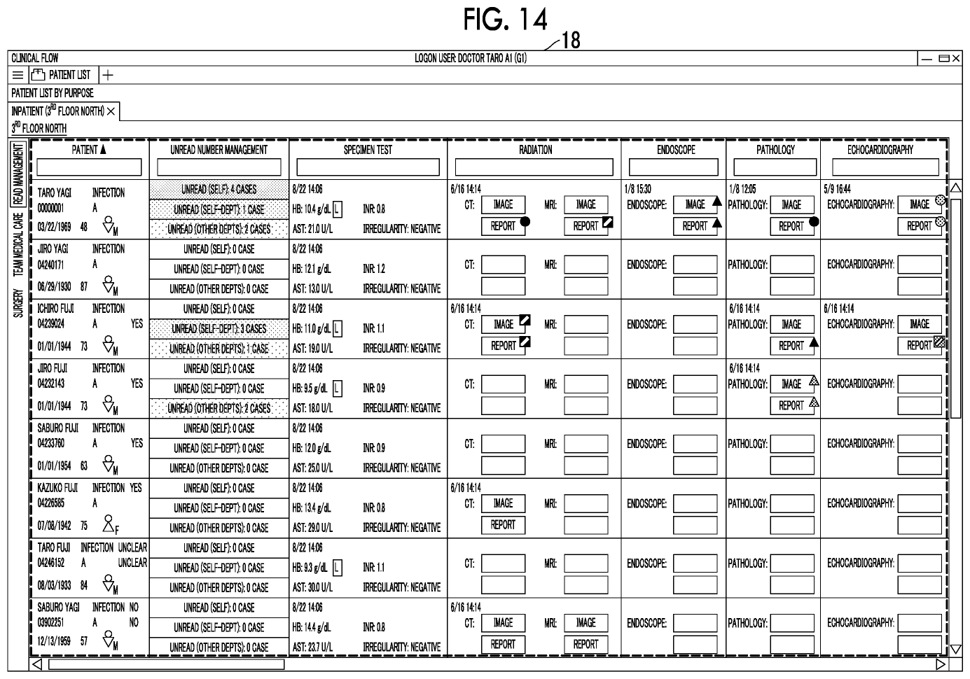

The conference information management unit 47 generates conference information at a predetermined specific timing. The conference information management unit 47 specifies the scheduled execution date of the conference with reference to the conference execution schedule 51. Then, as shown in FIG. 13, as a specific timing, for example, the server group 13 is accessed two days before the scheduled execution date of the conference, and the patient is extracted (Step S1). As described above, the server group 13 includes the electronic medical record server 21, and the conference information management unit 47 receives the electronic medical record 24, an order 27, or a medical care record 28 from the electronic medical record server 21. Except for the target patients of the previous NST conference, the target patients of the NST conference are patients who have been described in the electronic medical record 24 as requiring nutritional management in the last two weeks, or patients who have been issued a document of order 27 including the content of nutritional instructions and medical care record 28 including the content of nutritional management. For example, as shown in FIG. 13, the target patients of the NST conference are five patients, “Taro Fuji”, “Hanako Fuji”, “Jiro Fuji”, “Taro Yagi”, and “Saburo Yagi”. In a case where the medical care support device 11 can access the clinical flow 18, it is more efficient to extract the patient from the clinical flow 18. As shown in FIG. 14, the clinical flow 18 is a list of medical care processes of a plurality of patients generated based on the electronic medical record 24 or the medical report 26.

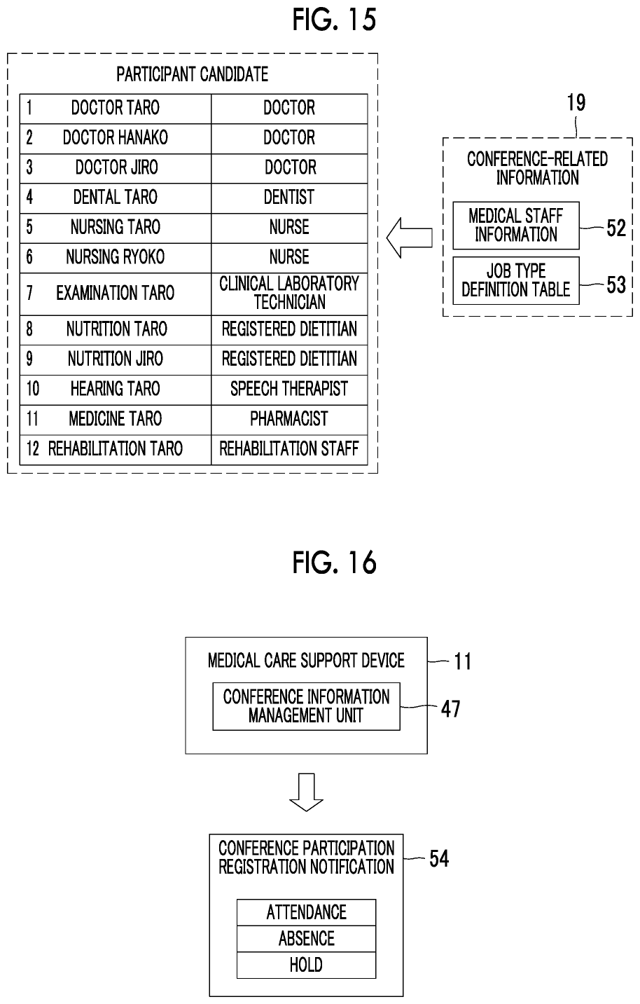

Further, the conference information management unit 47 extracts participant candidates of the conference from the medical staff information 52 by using the job type definition table 53 (Step S1). At the NST conference, referring to the job type definition table 53, eight job types of doctors, nurses, clinical laboratory technicians, registered dietitians, speech therapists, pharmacists, and rehabilitation staff are required or recommended to participate (see FIG. 10). Therefore, as shown in FIG. 15, the conference information management unit 47 has medical staff whose job types are doctors, nurses, clinical laboratory technicians, registered dietitians, speech therapists, pharmacists, and rehabilitation staff as participant candidates, and transmits a notification for participation registration (participation registration notification) to the client terminal 12 or the mobile terminal 54 of the participant candidates (Step S2).

For example, as shown in FIG. 15, participation registration notifications are transmitted to 12 persons of doctors “Doctor Taro”, “Doctor Hanako”, and “Doctor Jiro”, dentist “Dental Taro”, nurses “Nursing Taro” and “Nursing Ryoko”, clinical laboratory technician “Examination Taro”, registered dietitians “Nutrition Taro” and “Nutrition Jiro”, speech therapist “Hearing Taro”, pharmacist “Medicine Taro”, and rehabilitation staff “Rehabilitation Taro”.

As shown in FIG. 16, the participant candidate who has received the participation registration notification selects either “Attendance”, “Absence”, or “Hold” on each mobile terminal 54 or the like. The selection result is transmitted to the conference information management unit 47, and in the case where the conference information management unit 47 receives the result that the participant candidate has selected “Attendance”, the participant candidate is registered in the conference information as a participant of the conference (Step S3).

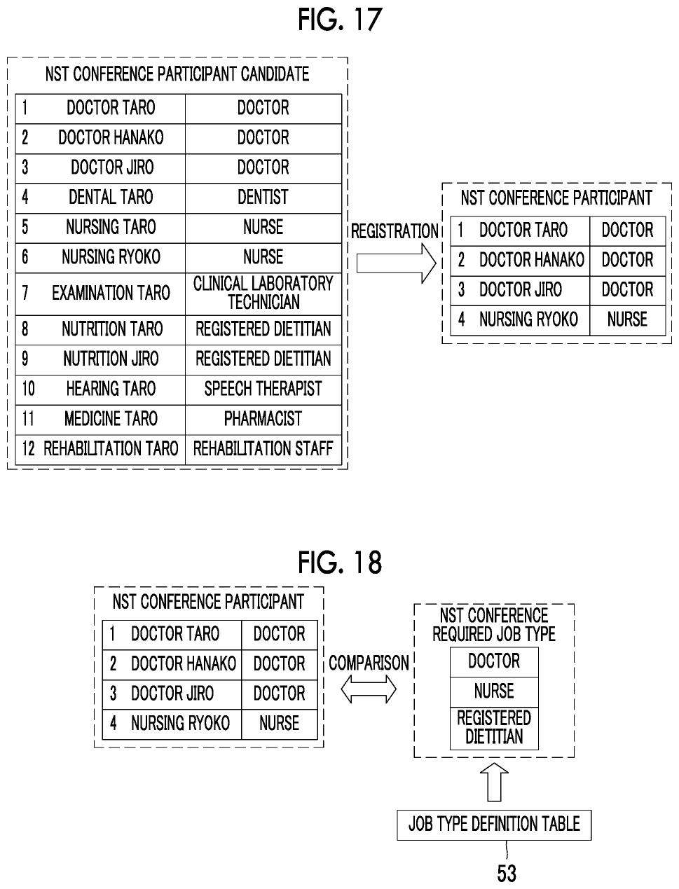

Furthermore, in a case where the conference information management unit 47 receives a result that the medical staff selects “Attendance”, the conference information management unit 47 generates conference information in which the type of conference, the execution date and time, the target patients, and the participants are associated with each other, based on target patients of the NST conference and selection results of participant candidates for the participation registration notification. For example, as shown in FIG. 17, in a case where there are 12 participant candidates, doctors “Doctor Taro”, “Doctor Hanako”, and “Doctor Jiro”, nurse “Nursing Ryoko”, clinical laboratory technician “Examination Taro”, registered dietitian “Nutrition Taro”, speech therapist “Hearing Taro”, pharmacist “Medicine Taro”, and rehabilitation staff “Rehabilitation Taro”, and the selection result of “participation” is transmitted to four of “Doctor Taro”, “Doctor Hanako”, “Doctor Jiro”, and “Nursing Ryoko” among these 12 participant candidates, these four people are registered as participants in the NST conference. In addition to the registered participants, the conference information display screen 17 displays the type, date and time, place, and target patient of the conference.

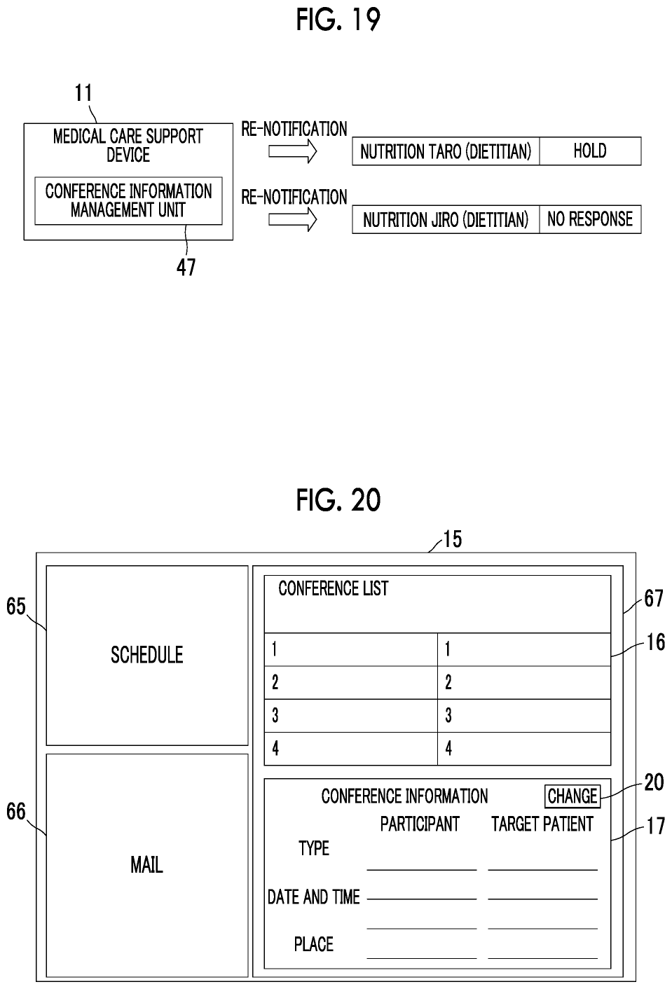

As shown in FIG. 17, there are four participants registered at the NST conference. As shown in FIG. 18, the participant job type determination unit 50 compares the job types of these four persons with the job types defined in the job type definition table 53 (Step S4). Doctors, nurses, and registered dietitians are required to participate in the NST conference. Therefore, since there is a shortage of registered dietitians, the determination result is NG. As shown in FIG. 19, in a case where there is a participant candidate who has not responded to the participation registration notification or has selected to hold the participation among the participant candidates who received the above-mentioned participation registration notification, the conference information management unit 47 transmits a participation registration notification (re-notification) to the terminal of the participant candidate again (Step S5). For example, the participation registration notification is transmitted again to the registered dietitian “Nutrition Taro” who has selected “Hold” and “Nutrition Jiro” who has not responded. Alternatively, a notification that the job type determination is NG may be transmitted to the terminal of the responsible person of the conference. In a case where the determination result is OK, it is not necessary to transmit the re-notification of participation registration.

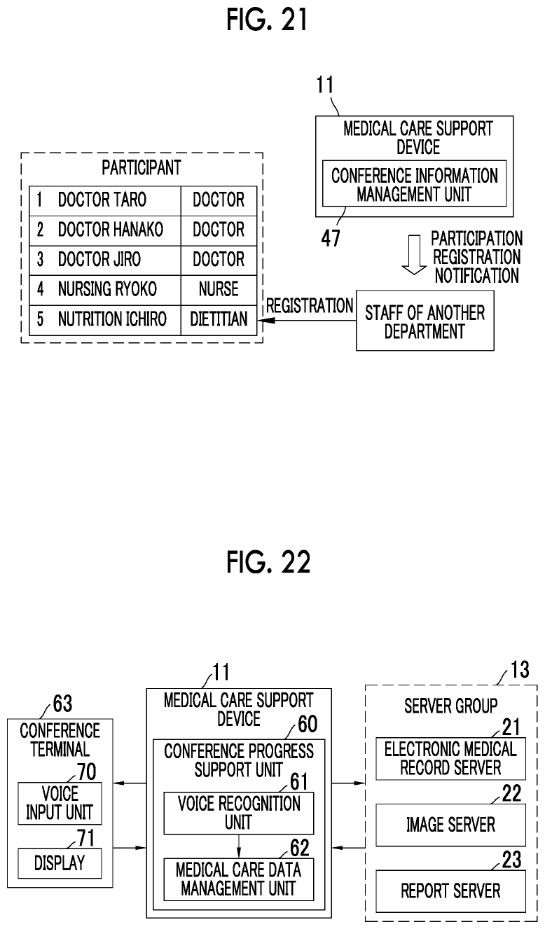

It is also possible to register the participation of medical staff other than participant candidates. As shown in FIG. 20, once a change button 20 on the conference information display screen 17 is clicked, the conference information display screen 17 displays that the conference information can be changed. Once any medical staff is added as a participant candidate, the added participant candidate is registered as a participant of the conference in a case where attendance is selected in response to the above-mentioned participation registration notification. For example, as shown in FIG. 21, in the case of additionally registering a registered dietitian of another department, “Nutrition Ichiro”, the participation registration notification is transmitted from the conference information management unit 47 to the terminal of “Nutrition Ichiro” by performing the additional registration operation on the conference information display screen 17, and in a case where “Nutrition Ichiro” who received the participation registration notification selects to participate on the terminal, he/she is registered as a participant of the conference. For input of medical staff for additional registration, after clicking the change button 20 on the conference information display screen 17, a list of medical staff that can be additionally registered may be displayed in a pop-up, and a selection may be made from the list.

In the present embodiment, an example in which the medical care support device 11 and the conference-related information database 11A are connected by the network 14 has been illustrated, but the present disclosure is not limited thereto. For example, in a case where the conference information management unit 47 generates the conference-related information 19, the conference-related information 19 may be held in the storage device 30A or the like of the medical care support device 11.

### Second Embodiment

Next, a second embodiment will be described. In the present embodiment, as shown in FIG. 22, the medical care support device 11 comprises a conference progress support unit 60. The conference progress support unit 60 functions as a voice recognition unit 61 and a medical care data management unit 62. Further, a conference terminal 63 (medical care data display terminal) that displays medical care data used in a conference being executed is provided with a voice input unit 70 (for example, a microphone or the like).

The voice recognition unit 61 transmits an instruction to transmit medical care data included in the voice to the server group 13 (medical care data storage unit) based on the voice input from the voice input unit 70, and the server group 13 transmits the medical care data instructed to be transmitted to the medical care data management unit 62. The medical care data management unit 62 displays the received medical care data on the conference terminal 63. For example, in a conference being executed, a participant who is proceeding with the conference inputs an instruction to acquire image data of the target patient to the conference terminal 63 in a voice, and based on the input voice, the medical care data management unit 62 acquires the instructed medical care data from the server group 13 and displays the medical care data on a display 71 of the conference terminal 63. Note that, the transmission instruction may be given by inputting the instruction term in characters at the conference terminal 63.

### Third Embodiment

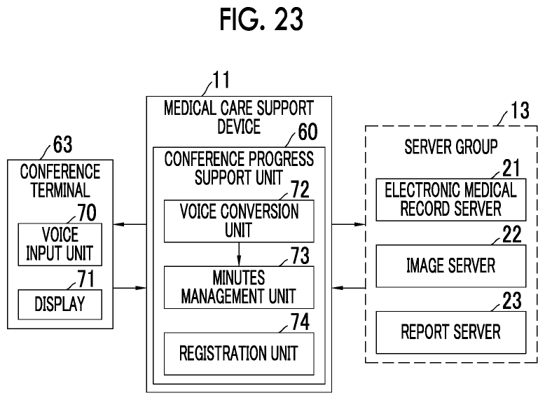

Next, a third embodiment will be described. In the present embodiment, as shown in FIG. 23, the conference progress support unit 60 functions as a voice conversion unit 72 and a minutes management unit 73. The voice conversion unit 72 converts the voice input from the voice input unit 70 into text information. The minutes management unit 73 creates conference minutes regarding the target patients of the conference based on the converted text information. A registration unit 74 accesses the electronic medical record server 21 of the server group 13 and registers the created conference minutes in the electronic medical record of the target patient.

In above embodiments, hardware structures of the processing units that execute various processes such as the conference information management unit 47, the participant job type determination unit 50, the conference progress support unit 60, the voice recognition unit 61, the medical care data management unit 62, the voice conversion unit 72, the minutes management unit 73, and the registration unit 74 are various processors as shown below. The various processors include a central processing unit (CPU) as a general-purpose processor functioning as various processing units by executing software (program), a programmable logic device (PLD) as a processor of which the circuit configuration can be changed after manufacturing such as a graphical processing unit (GPU) or a field programmable gate array (FPGA), a dedicated electrical circuit as a processor having a circuit configuration designed exclusively for executing various kinds of processing, and the like.

One processing unit may be configured by one of various processors, or may be configured by a combination of the same or different kinds of two or more processors (for example, a combination of a plurality of FPGAs, a combination of a CPU and an FPGA, a combination of a CPU and a GPU, or the like). In addition, a plurality of processing units may be configured by one processor. As an example of configuring a plurality of processing units by one processor, first, as represented by a computer, such as a client or a server, there is a form in which one processor is configured by a combination of one or more CPUs and software and this processor functions as a plurality of processing units. Second, as represented by a system on chip (SoC) or the like, there is a form of using a processor for realizing the function of the entire system including a plurality of processing units with one integrated circuit (IC) chip. Thus, various processing units are configured by using one or more of the above-described various processors as hardware structures.

More specifically, the hardware structure of these various processors is an electrical circuit (circuitry) in the form of a combination of circuit elements, such as semiconductor elements. The hardware structure of the storage unit is a storage device such as a hard disc drive (HDD) or a solid state drive (SSD).

The medical care support device of the present disclosure may employ an embodiment of the following processor device type.

A processor device causing a conference information management unit to generate, for each of conferences scheduled to be executed, conference information including information on a type of the conference, target patients for the conference, participants of the conference, and an execution date and time of the conference, a participant job type determination unit to compare a job type definition table in which participant job types for each type of the conferences are defined with the conference information to determine whether or not the participants of the job type defined in the job type definition table are included in the conference scheduled to be executed, and the conference information management unit to give a notification of a determination result of the participant job type determination unit.

### EXPLANATION OF REFERENCES

10: medical care support system

11: medical care support device

11A: conference-related information database

12: client terminal

13: server group

14: network

15: display screen

16: conference list display screen

17: conference information display screen

18: clinical flow

19: conference-related information

20: change button

21: electronic medical record server

21A: medical record database

22: image server

22A: image database

23: report server

23A: report database

24: electronic medical record

25: examination image

26: medical report

27: order

28: medical care record

30: storage device

30A: storage device

30B: storage device

31: memory

32: CPU

32A: CPU

32B: CPU

33: communication unit

34: display

34B: display

35: input device

35B: input device

36: data bus

40: viewer software

41: GUI control unit

42: request issuing unit

45: operation program

46: request reception unit

47: conference information management unit

48: display screen generation unit

49: distribution control unit

50: participant job type determination unit

51: conference execution schedule

52: medical staff information

53: job type definition table

54: mobile terminal

60: conference progress support unit

61: voice recognition unit

62: medical care data management unit

63: conference terminal

65: schedule display unit

66: mail display unit

67: conference information display unit

70: voice input unit

71: display

72: voice conversion unit

73: minutes management unit

74: registration unit

What is claimed is:

1. A medical care support device comprising
a processor configured to:
generate, for each of conferences scheduled to be executed, conference information including information on a type of the conference, target patients for the conference, participants of the conference, and an execution date and time of the conference;
compare a job type definition table in which participant job types for each type of the conferences are defined with the conference information to determine whether or not the participants of the job type defined in the job type definition table are included in the conference scheduled to be executed; and
give a notification of a determination result.

2. The medical care support device according to claim 1, wherein the processor is configured to generate a conference list display screen in which the conferences scheduled to be executed are listed.

3. The medical care support device according to claim 2, wherein the processor is configured to generate a conference information display screen that displays the conference information of the conference selected from the conference list display screen.

4. The medical care support device according to claim 3, wherein the processor is configured to receive a change operation for changing the conference information displayed on the conference information display screen, and change the conference information to the received contents.

5. The medical care support device according to claim 1, wherein the job type definition table is associated with participant job type importance, which is importance of the participant job type for each type of the conferences.

6. The medical care support device according to claim 1, wherein the processor is configured to extract the target patient for each type of the conferences by using medical care information related to medical care of patients, and generate the conference information of the conference for the extracted target patient.

7. The medical care support device according to claim 1, wherein the processor is configured to extract candidates of the participants for each type of the conferences by using the job type definition table, and transmit a participation registration notification to the candidates of the participants.

8. The medical care support device according to claim 1,
wherein the conferences are categorized by medical care processes, and
the participant job type includes a doctor and a nurse.

9. The medical care support device according to claim 1,
wherein the processor is configured to:
recognize a voice input from a microphone; and
in a case where the voice recognized during the execution of the conference includes an instruction to transmit medical care data acquired in medical care of a target patient of the conference being executed, access a server in which the medical care data is stored and acquire the medical care data; and
wherein the medical care support device further comprising:
a medical care data display terminal that displays the medical care data from the processor.

10. The medical care support device according to claim 9, wherein the processor is configured to:
recognize a voice input from the microphone;
convert the voice recognized during the execution of the conference into text information; and
create minutes of the conference being executed by using the converted text information.

11. The medical care support device according to claim 10, wherein the processor is configured to register the minutes of the conference in association with an electronic medical record of the target patient.

12. A medical care support method comprising
generating, for each of conferences scheduled to be executed, conference information including information on a type of the conference, target patients for the conference, participants of the conference, and an execution date and time of the conference;
comparing a job type definition table in which participant job types for each type of the conferences are defined with the conference information to determine whether or not the participants of the job type defined in the job type definition table are included in the conference scheduled to be executed; and
giving a notification of a determination result.

13. A non-transitory computer readable recording medium storing a medical care support program that causes a computer to perform:
generating, for each of conferences scheduled to be executed, conference information including information on a type of the conference, target patients for the conference, participants of the conference, and an execution date and time of the conference;
comparing a job type definition table in which participant job types for each type of the conferences are defined with the conference information to determine whether or not the participants of the job type defined in the job type definition table are included in the conference scheduled to be executed; and
giving a notification of a determination result.
